(19) United States
(12) Patent Application Publication
(10) Pub. No.: US 2026/0315192 A1
(43) Pub. Date: Oct. 15, 2026

(54) FIDUCIAL MARKER WITH MUA INTERFACE

(71) Applicants: Daniel Domingue, Lafayette, LA (US); Adam Hogan, Virginia Beach, VA (US); Keith Klaus, Flowood, MS (US)

(72) Inventors: Daniel Domingue, Lafayette, LA (US); Adam Hogan, Virginia Beach, VA (US); Keith Klaus, Flowood, MS (US)

(21) Appl. No.: 19/176,859

(22) Filed: Apr. 11, 2025

(51) Int. Cl. A61C 8/00; A61B 90/00; A61C 9/00

(52) U.S. Cl. CPC A61C 8/0001; A61B 90/39; A61C 9/004; A61B 2090/3912; A61B 2090/3916; A61B 2090/3991

(57) ABSTRACT

Disclosed is a fiducial marker for use in mapping the jaw anatomy of a patient. The fiducial marker includes a threaded implant portion and a cap thereon that includes an interface for a multi-unit abutment.

### FIELD OF THE INVENTION

The present invention relates to dental implant technology, specifically to a fiducial reference screw with a novel multi-unit abutment (MUA) interface. The invention is designed to facilitate precise alignment of jaw scans during dental implant procedures. The present invention enables improved accuracy and consistency in the placement and fitting of dental prosthetics by providing a stable and identifiable reference point for digital and physical scan alignment.

### BACKGROUND OF THE INVENTION

Dental implants are devices which are surgically implanted into the jawbone of a patient in the areas near to surgical sites. These devices are used as a temporary (only during surgery) fixated reference marker and serve to provide common reference point when stitching in CAD from preop to post op scans. This titanium self-tapping self-threading device is fixated into the jaw and removed immediately following the surgical procedure. In modern dental implantology, accurate alignment of jaw scans is critical for the proper fitting and functionality of dental prosthetics. Current methods often rely on conventional scan bodies or markers, which can introduce positional inaccuracies due to imprecise seating, rotational misalignment, or surface irregularities.

MUAs are commonly used in implant-supported prosthetics to create a stable connection between the implant and the restoration. However, existing MUA designs often lack dedicated fiducial surfaces or flat interface areas that would allow for reliable scan alignment.

The inventors identified a need for a more precise and repeatable alignment system, which led to the development of the present invention. This invention addresses the limitations of current systems by incorporating a flat MUA interface specifically designed to serve as a consistent reference point for digital and physical jaw scans. This enhancement significantly reduces scan misalignment errors and improves the overall accuracy of implant-supported restorations.

Dental prosthetics are typically made using computer-assisted design (CAD) tools and software. The use of CAD allows the surgeon to see the final product before anything is actually made so changes or adjustments can be made and displayed in real time. The digital precision of CAD provides consistent and reliable modeling.

Implants must be placed in a specific position and in alignment to the prospective teeth that they will eventually support. Therefore, the positioning of these dental implants must be precise to enable teeth to be made that will function properly and be esthetic in appearance.

In the world of dental implants, precision is everything. A perfectly placed implant not only ensures long-term success but also provides the comfort and aesthetics patients deserve. Techniques for creating accurate models of a patient's teeth can use analog or digital methods.

Accurate analog impression materials have been developed that allow for precise capture and duplication of the dentition and the restorative components, but this technology still requires a series of laborious workflows to produce analog models and prostheses. Physical impressions can also deform during handling which can lead to errors in the final restoration. Traditional impressions often cause discomfort for patients, particularly those with a sensitive gag reflex.

Digital CAD systems make extensive use of layers to build a model. This allows for additional positional alignment in a separate layer that uses selected reference points on the patient's existing teeth (which may later be removed as part of the restoration) in conjunction with the fiducial markers for building the preliminary model.

The process starts with an accurate scan of the patient's upper and lower jaw (the upper arch and the lower arch) to identify each tooth shape, position, and relative orientation. The scanning process can be done with a series of digitally joined x-ray images (photogrammetry) and/or an intra-oral scanner.

Photogrammetry is an advanced technique that captures precise 3D spatial data using high-resolution images that are then combined with appropriate computer software to create a digital model in 3D. The exact positions of dental implants can be determined with micrometer-levels of accuracy. Either two or four cameras can be used to capture the images. The photogrammetry technique is non-invasive and significantly reduces the need for additional appointments to ensure a proper fit.

Intraoral scanning is a procedure that uses a handheld device to create a digital impression of a patient's teeth and gums. The process includes (a) projecting a light source onto the teeth and gums of the patient, (b) capturing the resulting images with imaging sensors, and (c) stitching the thousands of captured images together to make a 3D model.

Both photogrammetry and intra-oral scanning require some type of reference marker that is mounted in the patient's mouth in a stable position. These reference markers are called “fiducial markers” and exhibit one or more visible features (e.g., a trilobed shape, a formed prominence, etc.) that uniquely show the position and orientation of the marker both before and after surgery. Those features are then used by the CAD system as points of reference to model the various components of the arch and design a proper fit for one or more implanted abutments.

A scan body with identifiable indicia is used to help make a digital model. For example, a scan body can be screwed into the dental implant and is scanned using a commercially available intra-oral scanner (IOS). The scan body can allow a CAD/CAM program to determine the orientation and angle of the scan body relative to the hexagonal shape on the dental implant, so that the CAD/CAM program can be used to design the prosthetic to be fitted precisely to the implant. See USD9612083 entitled “Scan Body.”

Fiducial markers exhibit one or more visible features (e.g., a trilobed shape, a formed prominence, etc.) that uniquely show the position and orientation of the marker both before and after surgery. Those features are then used by the CAD system as points of reference to model the various components of the arch and design a proper fit for one or more implanted abutments.

Obtaining a reference for natural head posture is important two-dimensionally and three-dimensionally to enable appropriate design and alignment of two-dimensional photographs as well as three dimensional facial scans. The reference points become the fixed locations from which all other shapes and positions are calculated.

See generally U.S. Pat. No. 5,967,777 (the disclosure of which is hereby incorporated by reference) that relates to the use of fiducial markers in CT scans for enhanced positional accuracy of the drilling and implanting processes. See also US Publication No. 2012/0214121 that discusses a technique for combining CT scan data with optical scan data for enhanced positional mapping.

It would be advantageous to have a fiducial marker that would reliably help to map the shape and contours of a patient's jaw and teeth and thereby enhance the accuracy of any prosthetic.

It would also be desirable to have a fiducial marker that could be connected to either a scan body or a photogrammetry interface so one device could be used with either technique or both techniques without discomfort or delays associated with either mapping process.

It would also be desirable to have a fiducial marker that could be used for a combination of both photogrammetry and intra-oral scanning with the same device and without having to remove any portion of that devices that has already been installed in the patient's jaw, even if temporarily for the mapping process.

The currently accepted technique uses separate products for the abutments and the fiducial markers. This separation of function adds steps to add, use, and then remove the fiducials independently of the abutments on which the prosthetics are attached.

It would be desirable to have tools and a workflow process that would reduce the number of steps involved with digitally mapping a patient's arch and attaching a suitably designed prosthetic.

One of the components frequently used in connection with dental prosthetics is an abutment for a variety of reasons.

An abutment connects a dental prosthetic (such as a crown) to a dental implant (typically a post made of titanium) inserted into a hole drilled into the jawbone. Once in position, it takes several months for the post to be stable enough to act as the root for the tooth and the abutment to connect the dental prosthetic.

A multi-unit abutment (MUA) is different from a regular abutment. An MUA is a specialized abutment commonly used with zygomatic dental implants and full arch replacements (all-on-four dental implants). An MUA is a specialized dental implant abutment designed to connect multiple implant fixtures to a single prosthesis and allows for correction of varying implant heights and angulation. An MUA addresses cases where two or more dental implants are placed near each other, typically when replacing multiple missing teeth in a row.

Multi-unit abutments are pivotal in enhancing the overall experience and outcomes for patients undergoing all-on-four dental implant surgeries. These components come in various sizes and angulations depending on the patient's needs and the type of dental implant used. The available angulations, which commonly include 0°, 17°, 30°, and 45°, with specialty sizes such as 52.5° and 60° in some cases, allow for a remarkable degree of flexibility during the placement of all-on-four implants.

One of the key benefits of MUAs is their ability to address slight misalignments in a patient's teeth. When these misalignments exist, strategically placing different MUAs with varying angles can level off the restoration, ensuring a more harmonious and natural-looking implant.

Height disparities in the jaw can also pose challenges when it comes to implants, and MUAs allow for the adjustment of abutment heights. With variations in implant heights, you can position lower implants above the gum tissue, while taller abutments can be placed deeper into the bone. This precision in aligning abutment heights creates a polished aesthetic appearance and evenly distributes any force exerted on the restoration from eating and speaking. Additionally, this height balance contributes to the longevity of the restoration, so patients have a durable and long-lasting solution for their dental needs.

Abutments for implant dentistry have multiple advantages. One is the ability to correct implant angulation. In cases where dental implants aren't perfectly aligned, MUAs can reposition them for a natural appearance and optimal functionality for the final prosthesis. MUAs also enable the placement of screw access holes in better locations, making the restoration process go a lot more smoothly. MUAs also allow different implant systems to be used in coordination with one another,

Another advantage is that the design of MUAs allows for clear visualization of prosthetic connections, leading to precise impressions. This improved accuracy translates into better-fitting restorations that are more comfortable. Additionally, the smaller screw access holes enhance aesthetics and simplify the task of managing the prosthesis by reducing the amount of material needed.

Due to MUAs establishing a level restorative platform among multiple implants, there is more of a uniform height and alignment among the abutments. It makes the implant's creation easier and contributes to long-term soft tissue maintenance around the implant sites. When replacing or adjusting the prosthesis, MUAs are easy to remove, causing minimal disturbance to surrounding soft tissues and minimizing patient discomfort.

MUAs are traditionally placed after the patient's implant site has healed with the healing abutment, and the permanent restoration is ready to be placed. The first step is to remove the healing abutment. Next, the selected abutment is placed into the implant before being checked for fit. Once the fit is confirmed, the abutment will be tightened in place.

The next thing to do is to capture an oral impression or dental scan of the mouth in order to obtain the shapes of the necessary prosthetic. In some cases, a traditional denture or bridge can be converted into an implant-supported denture or bridge.

It would be desirable to streamline the process of forming a stable implant platform for installation of a prosthetic,

### SUMMARY OF THE INVENTION

It is an object of the invention to provide an accurate fiducial marker.

It is also an object of the invention to streamline the workflow associated with the overall restoration process from mapping to restoration.

It is a further object of the invention to provide a fiducial marker that would fit a scan body or a photogrammetry interface, or both.

In accordance with these and other objects of the invention that will become apparent from the description herein, a fiducial marker according to the present invention comprises: (a) an implant portion comprising a self-tapping, helically-threaded screw body dimensioned for implant into bone, such as maxillary or mandibular bone, and (b) a cap on said screw body that comprises at least one pair of opposing, vertically flat regions that allow the screw body to be turned so advance or retract the threaded screw body and an interface thereon for a multi-unit abutment.

The fiducial marker of the present invention is very flexible to fit the needs of the doctor and the patent. For example, the MUA head on the present marker can be used to hold any MUA scan body as well as any photogrammetry interface without removing or reimplanting a suitable base for the other: one MUA works for both and provides compatibility with multiple scanning and photogrammetry technologies so it is more broadly useful. Also, the self-tapping design of the present marker means that the marker can be hand driven without the need for a pilot hole osteotomy.

The MUA head of the present invention provides the ability to attach any of the standard MUA components for significantly greater versatility compared to conventional fiducial markers. The MUA head allows for convenient integration with intraoral scanners and photogrammetry markers which allows for seamless digital workflows and improved precision in guided surgery and implant placement. Notably, the ability to keep the photogrammetry marker in place throughout the procedure allows continuous registration throughout surgery resulting in reduces positional errors and enhanced workflow efficiency.

Importantly, the present invention improves the workflow of dental restoration and prosthesis installation as well as the ability of the surgeon to align meshes in the CAD files and index the alignment with photogrammetry.

The present invention provides a fiducial reference screw featuring an integrated MUA interface with an identifiable surface designed for precise alignment of jaw scans.

Key features of the invention include: (a) a reference surface on the MUA interface, which provides a consistent and easily identifiable plane for scan alignment. A preferred reference surface is a flat, vertically upstanding surface; (b) compatibility with both digital and physical scanning systems to enhance and streamline accuracy during the design and manufacturing of implant-supported restorations; and (c) a fiducial reference function that reduces alignment discrepancies.

The present invention offers improved precision, reliability, and consistency in dental implant procedures regardless of the mapping method used. The design reduces common errors associated with scan misalignment, resulting in better prosthetic fit, improved patient outcomes, and increased efficiency for dental practitioners.

### BRIEF DESCRIPTION OF THE DRAWINGS

FIG. 1 is a front perspective view of the fiducial marker according to the invention.

FIG. 2 is a front view of the fiducial marker according to the invention.

FIG. 3 is a sectional view of a fiducial marker according to the invention with a scan body secured thereto.

FIG. 4 is an exploded parts view of the assembly shown in FIG. 3.

FIG. 5 is a view showing an exemplary placement of fiducial screws in a jaw.

### DETAILED DESCRIPTION

The fiducial marker of the present invention comprises: (a) an implant portion comprising a self-tapping, threaded screw body suitable for implant into bone, and (b) a transmucosal portion that comprises (i) a cap on said screw body and having pairs of opposing flat regions that allow the screw body to be turned so as to advance or retract the threaded screw body and (ii) an interface on said cap to receive a multi-unit abutment.

The implant part is screw-shaped and extends into the patient's maxillary or mandibular bone. The implant part is totally buried and invisible.

The term “implant abutment,” as used herein, generally refers to a component that is attached to the head of a dental implant for the purpose of securing, supporting or retaining the dental prosthesis. Typically, an abutment is a small metal connector piece that is used to join a dental implant to a dental prosthetic. Abutments also make it possible to switch out a dental prosthetic without needing to remove the entire implant.

A multi-unit abutment (“MUA”) interface for the present invention is a type of abutment interface that allows for the connection of multiple implant fixtures to a single prosthesis.

The fiducial marker of the present invention is preferably made from titanium or a titanium alloy

Of particular interests are titanium alloys. The main alloy used is so-called commercially pure titanium, cpTi. This metal is available in four grades numbered 1 to 4, according to the purity and the processing oxygen content. These grades differ in corrosion resistance, ductility and strength. Grade 4 cp-Ti has the highest oxygen content (around 0.4%) and best overall mechanical strength so it is most widely used for dental implants.

Titanium 6AL4V (grade 5 titanium) and 6AL4V ELI are alloys containing 6% aluminum and 4% vanadium. They are two of the most common types of titanium used in medicine and offer greater fracture-resistance when used in dental implants. Also known as Gr. 5 and Gr. 23, these are some of the most familiar and readily available types of titanium in the United States. See Table 1.

TABLE 1
Composition and properties of titanium alloys used as implants.

| | cpTi Grade | cpTi Grade | cpTi Grade | cpTi Grade | |
|---|---|---|---|---|---|
| | 1 | 2 | 3 | 4 | Ti6Al4V |
| Titanium | ca 99% | ca 99% | ca 99% | ca 99% | 90% |
| Oxygen | 0.18% | 0.25% | 0.35% | 0.4% | 0.2% max |
| Iron | 0.2% | 0.2% | 0.2% | 0.3% | 0.25% |
| Nitrogen | 0.03% | 0.03% | 0.05% | 0.05% | — |
| Hydrogen | 0.15% | 0.15% | 0.15% | 0.15% | — |
| Carbon | 0.1% | 0.1% | 0.1% | 0.1% | — |
| UTS/MPa | 240 | 340 | 450 | 550 | 900 |
| Yield | | | | | |
| strength/MPa | 170 | 275 | 380 | 480 | 850 |
| Elongation at | 25 | 20 | 18 | 15 | 10 |
| failure/% | | | | | |

The invention is conveniently described in the context of the embodiment shown in the figures. It should be understood that the same structures will be noted using the same reference numbers in the various view. It will also be understood that the device of the present invention and method if its use are applicable to any species having teeth. Preferably, the device and method are used for dentistry of mammals generally and humans in particular.

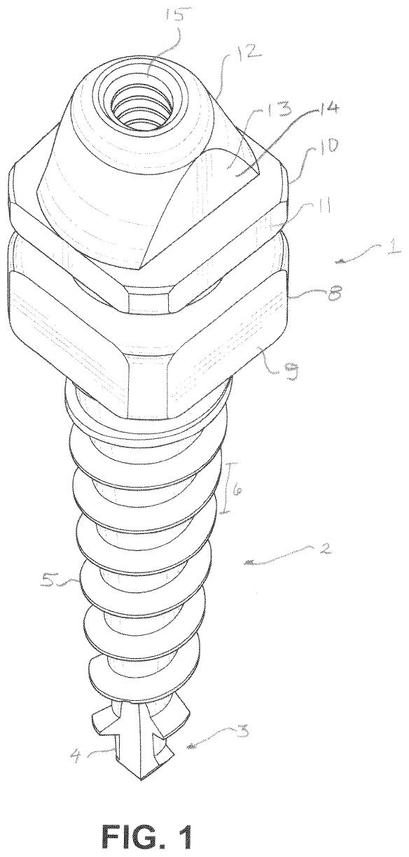

FIG. 1 shows a fiducial marker 1 according to the present invention with an MUA interface at the head thereof. Marker 1 is made with a tapered, threaded, implant portion 2 having a sharp leading edge 3 and self-tapping, thread 4 around leading edge 3. Self-tapping threads 4 facilitate the advance of marker 1 into position without the need for an implant hole/osteotomy. Preferably, marker 1 is installed at a position where a dental implant would not normally go so that marker 1 can be removed entirely after the surgery is complete and all scans or photos have been taken.

Threaded implant portion 2 has a tapering helical thread 5 from the bottom of cap 7 to leading edge 3. Tapering helical thread 5 is preferably at a pitch within a range sufficient to grip and become secured with bone to become properly ossified. Preferably, thread 4 has a pitch 6 from about 5 threads per inch (tpi) to about 30 tpi with a diameter within a range from about 0.1 mm to about 5 mm.

Cap 7 is integrally formed with the top of threaded portion 2 and has a first gripping section 8 characterized by pairs of opposing first flat, vertical surfaces 9 that can be used to turn marker 1.

Cap 7 also has a second gripping section 10 that also includes pairs of opposing second parallel flat surfaces 11 and an MUA interface 12 having a third gripping section 13 with one pair of vertically extending flat surfaces 14.

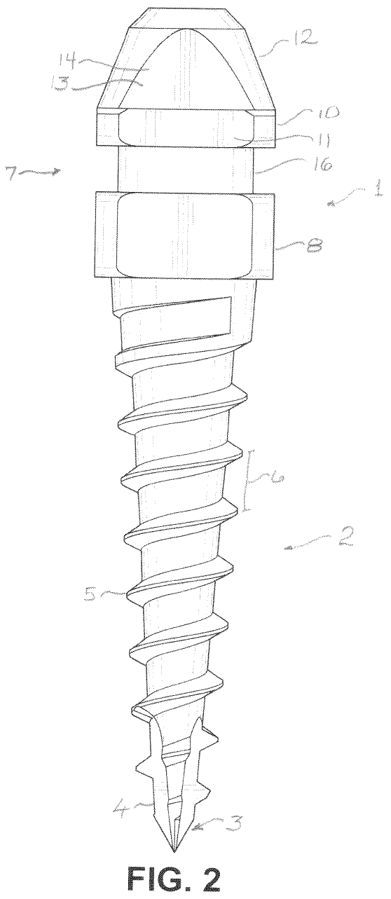

MUA interface 12 is on the top of second gripping section 10 and exhibits a generally frustoconical profile (shown better in FIG. 2) that can receive a variety of abutment screws and MUA devices in centrally-located, threaded opening 15 having a pitch and thread depth suitable to receive an abutment screw (not shown). A suitable abutment screw is described in my pending application Ser. No. 18/456,633, the disclosure of which is hereby incorporated by reference. Of particular interest are attachments to MUA interface 12 that serve as a scan body for intra-oral scanning and photogrammetry markers that facilitate the accurate mapping of the jawline arch and the tooth placement therein.

Between cap 7 and second gripping section 10 is a spacer 16 that allows MUA interface 12 to be positioned above the patient's jawline at a selected distance. Spacer 16 preferably has length within a range from about 1 mm to 20 mm, more preferably within a range from about 2 mm to 5 mm.

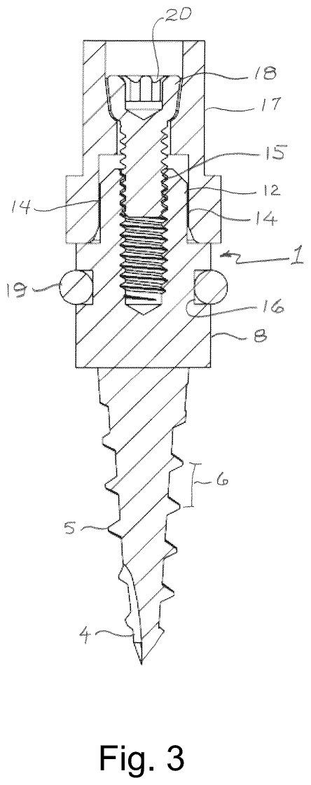

FIG. 3 illustrates a sectional view of the fiducial marker 1 of the present invention when combined with scan body 17 and abutment screw 18. O-ring 19 is disposed within spacer 16 to assist with the seal of marker 1 in the patient's jaw (not shown). Shaped recess 20 in abutment screw 18 interacts with a driver tool (not shown) to advance or retract abutment screw 18 within threaded opening 15 in MUA 12.

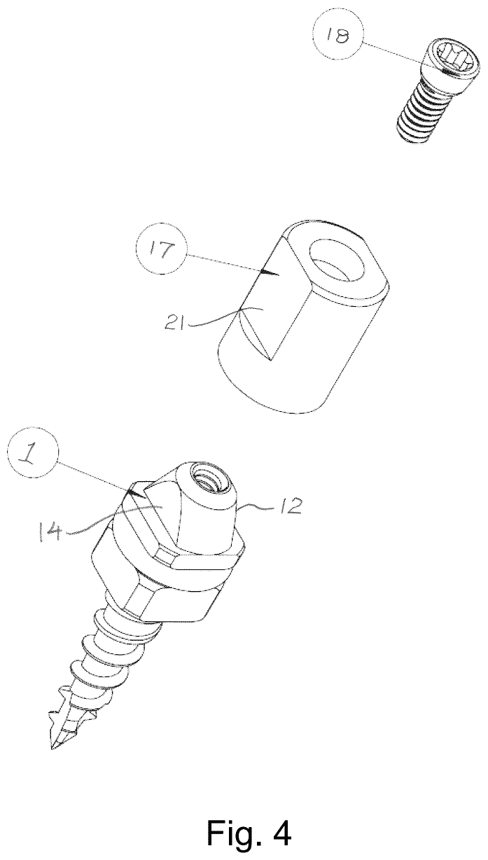

As shown in FIG. 4, MUA interface 12 on fiducial marker 1 serves as a support platform for scan body 17 which is secured in place with abutment screw 18. Vertical flat surfaces 14 can be used as reference indicia for scan mapping. Alternatively, scan body 17 exhibits one or more surface mapping indicia 21 whose location and orientation can be used in a digital scan of the patient's mouth to identify a position, height, and rotation relative to other mapping indicia on other scan bodies within the oral cavity.

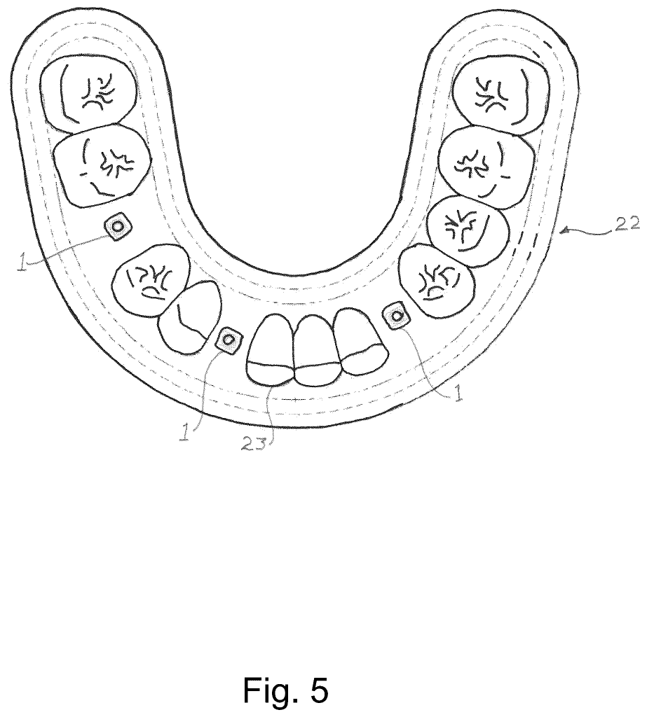

Once positioned within the patient's jaw 22 among the existing teeth 23 and mapped (see FIG. 5), scan body 17 is readily removed and replaced with a prosthetic (not shown) using abutment screw 18. This flexibility allows the use of both intra-oral scamming and photogrammetry markers for mapping of a patient's jaw without the need for a separate implant procedure. Just unscrew the abutment and swap out the type of marker to be used for the procedure. Fiducial marker 1 need not be removed from its implanted position during this process. This flexibility helps to streamline the reconstruction process and reduces inflammation to the patient's jaw that would otherwise be caused by removal of implanted port 2 or a separate healing implant (not shown).

Fiducial marker 1 is used by an oral cavity and jaw contour mapping process that includes: (a) installing fiducial marker 1 into the jaw of a patient's oral cavity, wherein said fiducial marker comprises: (i) a tapered, threaded implanting portion at a bottom of said implant and (ii) a multi-unit abutment disposed on top of said implant portion, wherein said fiducial marker 1 is installed at a position where implant screws configured to secure a permanent dental prosthetic will not be placed, (b) affixing to said multi-unit abutment a mapping device having a detectable indicia that is configured to be detectable with computer-aided cavity mapping by photogrammetry or intra-oral scanning techniques, (c) detecting a relative position of said detectable indicia within said cavity, and (d) creating a dimensional model of the patient's oral cavity and the contours of the patient's jaw within said cavity.

A photogrammetry marker and/or an intra-oral scanning marker can be used in the present process without removing the fiducial implant portion from the patient's jaw. This allows a degree of flexibility for the surgeon when planning the mapping process and also afford an opportunity to use both photogrammetry and intra-oral scanning to build a hybrid model of the patient's oral cavity.

Preferably, the fiducial implant is removed from the patient's jaw when the mapping process is done. The fiducial implant hole in the patient's jaw is small (preferably about 1.5-2.5 mm) and will naturally heal closed.

The dimensional model resulting from the mapping process is used by a prosthetic CAD system to configure a suitable prosthetic and its position-securing implants.

1. A fiducial marker that comprises: (a) an implant portion comprising a self-tapping screw body dimensioned for implant into bone, and (b) a cap on said screw body that comprises (i) pairs of opposing, vertically flat regions that allow the screw body to be turned so advance or retract the threaded screw body and (ii) a multi-unit abutment interface thereon.

2. A fiducial marker according to claim 1 wherein said implant portion has a pitch from about 5 threads per inch (tpi) to about 30 tpi with a diameter within a range from about 0.1 mm to about 5 mm.

3. A fiducial marker according to claim 1 wherein said implant portion and said cap are integral.

4. A fiducial marker according to claim 1 wherein said multi-unit abutment interface comprises a threaded top opening dimensioned to receive an abutment screw.

5. A fiducial marker according to claim 1 wherein said multi-unit abutment interface exhibits a generally frustoconical profile.

6. An oral cavity mapping process that includes: (a) installing a fiducial marker into the jaw of a patient's oral cavity, wherein said fiducial marker comprises: (i) a tapered, threaded implanting portion at a bottom of said implant and (ii) a multi-unit abutment disposed on top of said implant portion, wherein said fiducial marker is installed at a position where implant screws configured to secure a permanent dental prosthetic will not be placed, (b) affixing to said multi-unit abutment a mapping device having a detectable indicia that is configured to be detectable with computer-aided cavity mapping by photogrammetry or intra-oral scanning techniques, (c) detecting a relative position of said detectable indicia within said cavity, and (d) creating a dimensional model of the patient's oral cavity and the contours of the patient's jaw within said cavity.
